(19) United States
(12) Patent Application Publication
(10) Pub. No.: US 2024/0382665 A1
(43) Pub. Date: Nov. 21, 2024

(54) ANTI-BACKFLOW NASAL ASPIRATOR

(71) Applicant: Yuyu Cao, Shenzhen (CN)

(72) Inventor: Yuyu Cao, Shenzhen (CN)

(21) Appl. No.: 18/203,768

(22) Filed: May 31, 2023

(30) Foreign Application Priority Data
May 20, 2023 (CN) 2023105735145

(51) Int. Cl. A61M 1/00

(52) U.S. Cl. CPC A61M 1/78; A61M 1/80; A61M 2205/8206; A61M 2210/0618

(57) ABSTRACT

An anti-backflow nasal aspirator, comprising: a housing, wherein a control assembly and a suction assembly are installed in the housing, and the suction assembly is opened or closed by the control assembly; a collection assembly, hermetically installed on the housing, and the collection assembly is hermetically docked with the suction assembly; the collection assembly is provided with a collection cavity; a suction nozzle, installed at the input end of the collection assembly; a diversion bracket, installed in the collection assembly. The nasal mucus is diverted through the diversion bracket to prevent the nasal mucus from entering the air passage and causing a short circuit of the main board, and through the structure of the joint between the diversion bracket and the collection cup mask, it can effectively prevent nasal mucus from diverting, which is more hygienic.

### 1. TECHNICAL FIELD

The invention relates to the technical field of nasal aspirators, in particular to an anti-backflow nasal aspirator.

### 2. BACKGROUND ART

Due to the serious air pollution in the city and the increasing number of children with nasal allergies, nasal secretions are mixed with dust, which will form boogers after drying and block the nasal passages; when a child has a cold, it is difficult to suck out the liquid secretion in the nasal cavity, and it is also difficult to remove the solid secretion in the nasal cavity, resulting in unsmooth breathing; in order to solve this problem, someone invented a nasal aspirator, through which the nasal mucus in the nasal cavity is sucked out.

However, when the current nasal aspirator is in use, the air pump is connected with the suction nozzle; when the nasal mucus is inhaled, since the cavity storing the nasal mucus is communicated with the air path of the entire air pump, the nasal mucus in the storage cavity can easily enter the air path of the air pump, which causes the air pump to be blocked and results in the failure of the main board and the air pump.

In order to allow nasal mucus to enter the channel storage cavity, a gap cavity needs to be formed at the docking joint of the collection cup mask of the suction nozzle for the nasal mucus to enter; due to the unreasonable structural design of traditional products, the liquid inlet gap is relatively large, which makes the nasal mucus stored in the nasal aspirator easy to escape to the outside through the liquid inlet gap when the nasal aspirator is not in use, which is very unhygienic.

### 3. SUMMARY OF THE INVENTION

In order to solve the above problems, the invention provides an anti-backflow nasal aspirator. Through the design and structure of the diversion bracket, the problem of nasal mucus entering the airway and damaging the air pump and the main board can be effectively solved, as well as the problem of liquid leakage when not in use.

The invention is realized by the following technical solutions: an anti-backflow nasal aspirator, comprising:
a housing, wherein a control assembly and a suction assembly are installed in the housing, and the suction assembly is opened or closed by the control assembly;
a collection assembly, wherein the collection assembly is hermetically installed on the housing, and the collection assembly is hermetically docked with the suction assembly; the collection assembly is provided with a collection cavity;
a suction nozzle, wherein the suction nozzle is installed at the input end of the collection assembly; when the suction assembly generates negative pressure, it acts on the suction nozzle;
a diversion bracket, wherein the diversion bracket is installed in the collection assembly; a diversion surface is formed on the diversion bracket, and the diversion surface is used for diverting nasal mucus to prevent nasal mucus from entering the airway of the suction assembly.

Preferably, the collection assembly comprises a collection cup mask and a collection cup base; the collection cup mask is connected and assembled with the collection cup base in a detachable manner, and an O-ring is installed on the assembly surface; the diversion bracket is installed between the collection cup mask and the collection cup base; one end of the collection cup base is connected to the suction assembly, and the other end thereof is sealed to a first docking port of the diversion bracket; a second docking port at the other end of the diversion bracket is docked with the inner docking joint of the collection cup mask.

Preferably, the diversion bracket comprises a columnar bracket body; one side of the bracket body forms a first docking port, and the other side of the bracket body is provided with a docking boss; the side of the docking boss opposite to the inner docking joint forms a second docking port, and the second docking port forms a liquid inlet gap cavity after being docked with the inner docking joint.

Preferably, the outer end of the collection cup mask is provided with an outer docking joint, and the outer docking joint is used for hermetically docking with the suction nozzle.

Preferably, the upper and lower ends of the diversion bracket are also provided with a diversion cover, and the whole diversion cover is diamond-shaped; inclined diversion surfaces are formed on both sides of the diversion cover; an air passage is formed in the diversion bracket, and the air passage is communicated with the suction assembly; at least one suction hole is provided at the position where the air passage is located at the diversion cover; the suction hole is located at the bottom of the diversion cover, and the suction hole is isolated from the inside of the collection assembly by the diversion cover.

Preferably, a collection cavity is formed after the collection cup mask is docked with the collection cup base through the diversion bracket, and the nasal mucus entering through the liquid inlet gap cavity enters into the collection cavity for collection.

Preferably, the housing comprises a front cover and a rear cover, which are assembled into one body; the housing is provided with a display screen lens; the control assembly is installed in the internal space formed by the front cover and the rear cover, and the control assembly comprises a main board and a lithium battery; the lithium battery is used to supply power to the main board and the suction assembly, and a display screen and a USB charging stand are installed on the main board.

Preferably, the main board is also installed with a button assembly; the housing is also installed with a horn, and the horn is connected to the main board and powered by the lithium battery.

Preferably, the suction assembly comprises an air pump and an air pump connecting seat; the air pump is connected to the main board, and is controlled to open and close by the main board; the air pump is powered by the lithium battery; the air pump is docked with the air pump connecting seat, and the other end of the air pump connecting seat is docked and sealed with the collection cup base.

Preferably, a body decoration ring is also installed outside the docking position of the air pump connecting seat and the collection cup base.

The beneficial effects of the invention are: the invention is installed with a diversion bracket between the collection cup base and the collection cup mask, and a diversion cover is provided on the diversion bracket, forming a diversion surface outside the diversion cover; in this way, the nasal mucus will flow over the diversion surface, avoiding the position of the airway, which effectively prevents the nasal mucus from entering the airway through the diversion bracket and causing damage to the air pump and the main board; Since the diversion bracket of the invention is provided with a docking boss, which is cooperated with the inner docking joint at the inner end of the collection cup mask, so that the liquid inlet gap cavity is provided with multiple inflection surfaces, and the gap can be effectively reduced; since multiple turning surfaces are provided, with the cooperation of a small gap, the nasal mucus in the collecting cavity can be effectively prevented from flowing out from the liquid inlet gap cavity when not in use, and the use is more hygienic.

### 4. BRIEF DESCRIPTION OF ACCOMPANY DRAWINGS

In order to explain the embodiments of the invention or the technical solutions in the prior art more clearly, the drawings that need to be used in the description of the embodiments or the prior art will be introduced hereinafter. Obviously, the drawings in the following description are only some embodiments of the invention. For those of ordinary skill in the art, other drawings may be obtained from these drawings without creative efforts.

FIG. 1 is an exploded diagram according to the invention;

FIG. 2 is an overall structural diagram according to the invention;

FIG. 3 is an internal sectional view according to the invention;

FIG. 4 is a schematic diagram of the internal structure according to the invention after removing the housing;

FIG. 5 is a schematic structural view of the diversion bracket according to the invention;

FIG. 6 is a partial enlarged view of place A in FIG. 3 according to the invention.

### DESCRIPTION OF REFERENCE SIGNS

1 refers to the suction nozzle; 2 refers to the collection cup mask; 3 refers to the diversion bracket; 4 refers to the collection cup base; 5 refers to the body decoration ring; 6 refers to the air pump connecting seat; 7 refers to the rear cover; 8 refers to the lithium battery; 9 refers to the air pump; 10 refers to the USD charging stand; 11 refers to the horn; 12 refers to the button assembly; 13 refers to the front cover; 14 refers to the display screen lens; 15 refers to the main board; 16 refers to the collection cavity; 31 refers to the bracket body; 32 refers to the first docking port; 33 refers to the diversion surface; 34 refers to the suction hole; 35 refers to the docking boss; 36 refers to the second docking port; 37 refers to the diversion cover; 38 refers to the liquid inlet gap cavity; 100 refers to the housing.

### 5. SPECIFIC EMBODIMENT OF THE INVENTION

All the features disclosed in the specification, or all disclosed methods or steps in the process, except for mutually exclusive features and/or steps, can be combined in any manner. Any feature disclosed in the specification (including any appended claims, abstract and drawings), unless specifically stated, can be replaced by other equivalent or equivalent alternative features. That is, unless otherwise stated, each feature is just one example of a series of equivalent or similar features.

In the description of the invention, it needs to be understood that the orientation or positional relationship indicated by the terms “one end”, “the other end”, “outer”, “upper”, “inner”, “horizontal”, “coaxial”, “center”, “end”, “length”, “outer end”, etc. are based on the orientation or positional relationship shown in the drawings, which are only for the convenience of describing the invention and simplifying the description, rather than indicating or implying that the device or element referred to must have a specific orientation, be constructed and operated in a specific orientation, and therefore cannot be understood as a limitation of the invention.

In addition, in the description of the invention, “plurality” means at least two, such as two, three, etc., unless otherwise specifically defined.

The terms such as “upper”, “above”, “lower”, “below” and the like used in the invention to indicate a relative position in space are used to describe the relationship of one unit or feature relative to another unit or feature as shown in the drawings for the purpose of facilitating explanation. The term of the relative position in space may be intended to include different orientations of the device in use or operation other than those shown in the figures. For example, if the device in the figure is turned over, the unit described as being “lower” or “below” other units or features will be “above” the other units or features. Therefore, the exemplary term “below” can encompass both above and below orientations. A device can be oriented in other ways (rotated by 90 degrees or other orientations), and the space-related descriptors used in this article are explained accordingly.

In the invention, unless otherwise clearly defined and limited, the terms “provided”, “sleeved”, “connected”, “through”, “plug” and other terms should be interpreted broadly; for example, it can be a fixed connection, it can be a detachable connection, or integrated; it can be a mechanical connection or an electrical connection; it can be a direct connection, or an indirectly connection through an intermediate medium, and it can be an internal communication between two elements or the interaction relationship between two elements, unless specifically defined otherwise. For those of ordinary skill in the art, the specific meaning of the above terms in the invention can be understood according to specific circumstances.

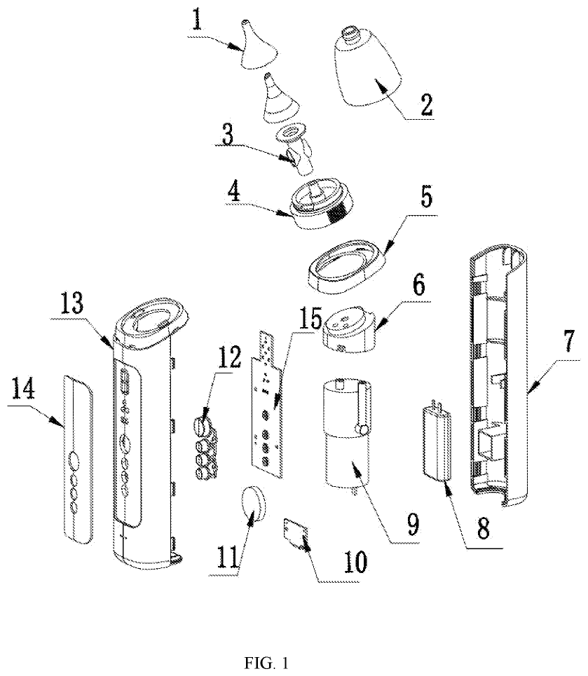

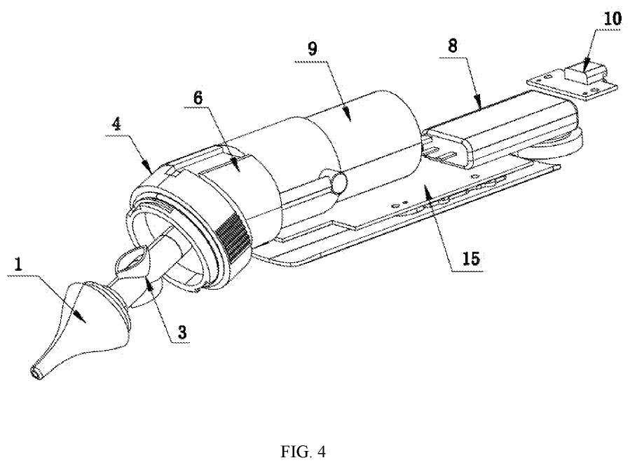

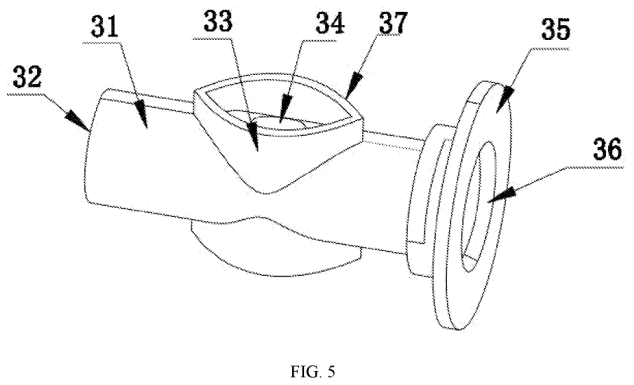

As shown in FIGS. 1 and 4, an anti-backflow nasal aspirator of the invention, comprising: a housing 100, wherein a control assembly and a suction assembly are installed in the housing 100, and the suction assembly is opened or closed by the control assembly; the suction assembly is used to generate negative pressure suction, and then achieve the purpose of negative pressure adsorption of nasal mucus; the control assembly can be opened or closed to control the suction pressure value;
it further comprises a collection assembly, wherein the collection assembly is hermetically installed on the housing 100, and the collection assembly is hermetically docked with the suction assembly; the collection assembly is provided with a collection cavity 16; the liquid sucked in by the suction assembly, such as nasal mucus, can enter the collection cavity 16 for collection, and can be cleaned regularly after collection, and can be cleaned by disassembling the collection assembly during cleaning;
in order to achieve the suction of nasal mucus, in the embodiment, it further comprises a suction nozzle 1, wherein the suction nozzle 1 is installed at the input end of the collection assembly; when the suction assembly generates negative pressure, it acts on the suction nozzle 1; the head of the suction nozzle 1 can be tapered, and the overall shape is long and thin, which is mainly used to extend the suction nozzle 1 into the nasal cavity, and achieve the purpose of sucking the nasal mucus after contacting the nasal mucus; the suction nozzle 1 can be detachably connected with the suction assembly through threads; the suction nozzle 1 can be replaced regularly, and different shapes can be replaced, such as the length and outer diameter of the suction nozzle 1, etc.; different age groups of users can use different types of suction nozzles 1; when the nasal aspirator is not in use, the suction nozzle 1 can be removed for separate storage;
in order to achieve the docking of the collection assembly and the suction assembly, in the embodiment, it further comprises a diversion bracket 3, wherein the diversion bracket 3 is installed in the collection assembly; a diversion surface 33 is formed on the diversion bracket 3, and the diversion surface 33 is used for diverting nasal mucus to prevent nasal mucus from entering the airway of the suction assembly; as shown in FIG. 5, the diversion surface 33 in the embodiment is inclined as a whole, which can allow the nasal mucus to flow smoothly on both sides; since the diversion surface 33 is provided, the nasal mucus will not enter the air passage, which effectively avoids damage to the air pump 9 and the main board 15, making it safer to use; the structure of the diversion surface 33 can be various, as long as it can complete the isolation and diversion of nasal mucus.

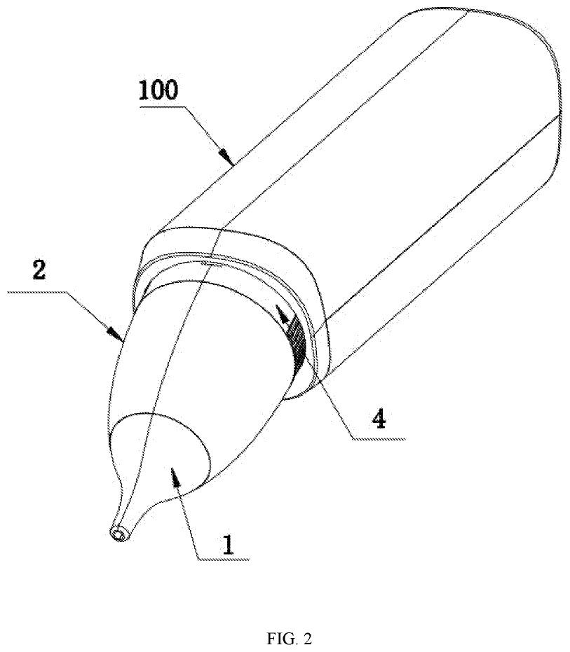

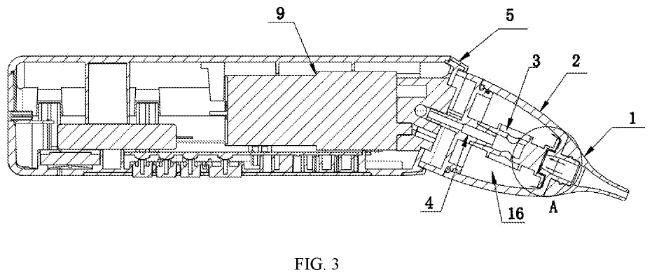

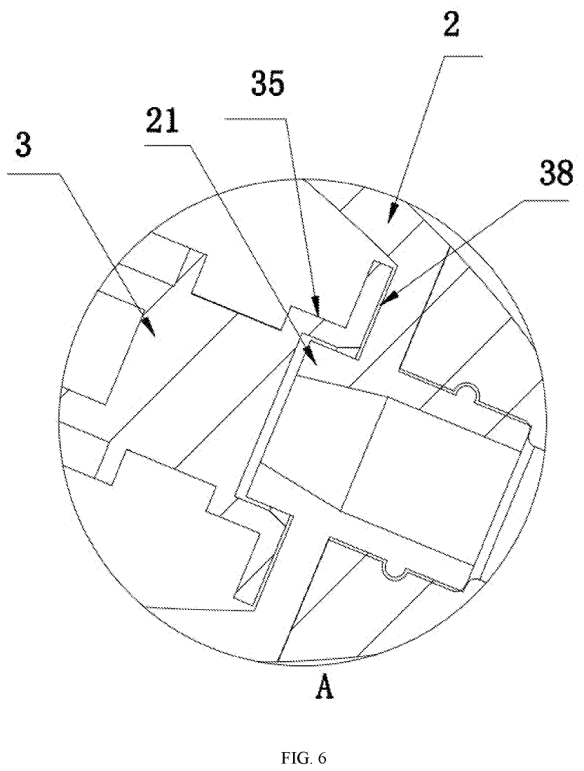

As shown in FIGS. 2 and 3, the collection assembly comprises a collection cup mask 2 and a collection cup base 4; the collection cup mask 2 is connected and assembled with the collection cup base 4 in a detachable manner; in order to increase the sealing performance of the collection cup mask 2 and the collection cup base 4 after assembly, and to prevent nasal mucus from flowing out, in the embodiment, an O-ring is installed on the assembly surface; the O-ring can be provided with multiple, or only one; the collection cup mask 2 and the collection cup base 4 can be connected by threads; when the two are locked to each other, the O-ring on the assembly surface is compressed to achieve the purpose of sealing; the diversion bracket 3 is installed between the collection cup mask 2 and the collection cup base 4; one end of the collection cup base 4 is connected to the suction assembly, and the other end thereof is sealed to a first docking port 32 of the diversion bracket 3; a second docking port 36 at the other end of the diversion bracket 3 is docked with the inner docking joint of the collection cup mask 2, as shown in FIG. 6

As shown in FIG. 5, the diversion bracket 3 comprises a columnar bracket body 31; one side of the bracket body 31 forms a first docking port 32, and the other side of the bracket body 31 is provided with a docking boss 35; the side of the docking boss 35 opposite to the inner docking joint forms a second docking port 36, and the second docking port 36 forms a liquid inlet gap cavity 38 after being docked with the inner docking joint. As shown in FIGS. 3 and 6, since a docking boss 35 is provided, and the docking boss 35 is provided with a second docking port 36 on the side opposite to the inner docking joint, after the inner docking joint is docked into the second docking port 36, a liquid inlet gap cavity 38 can be formed, and the liquid inlet gap cavity 38 is provided with a plurality of inflection surfaces, as shown in FIG. 6; therefore, through multiple inflection surfaces with small gaps, when the device is not in use, it is difficult for nasal mucus to flow out from the inflection surfaces with multiple small gaps, so as to prevent nasal mucus from overflowing.

The inhalation direction of nasal mucus is shown by the arrow in FIG. 3; when the suction nozzle 1 is inserted into the nasal cavity, the air pump 9 works to generate negative pressure suction to the suction nozzle 1, the suction nozzle 1 sucks the nasal mucus in the nasal cavity, and the nasal mucus passes through the liquid inlet gap cavity 38 and finally enters the collection cavity 16 for collection.

In order to allow the suction nozzle 1 to be docked with the outside of the collection cup mask 2, in the embodiment, the outer end of the collection cup mask 2 is provided with an outer docking joint, and the outer docking joint is used for hermetically docking with the suction nozzle 1. The suction nozzle 1 and the outer docking joint can be assembled by threads, and a sealing ring can also be installed on the assembly surface to improve the sealing effect, so as to prevent the nasal mucus from being unable to be sucked due to poor air tightness.

In order to isolate the nasal mucus so that the nasal mucus can flow along the diversion bracket 3 without entering the airway, in the embodiment, the upper and lower ends of the diversion bracket 3 are also provided with a diversion cover 37, and the whole diversion cover 37 is diamond-shaped; inclined diversion surfaces 33 are formed on both sides of the diversion cover 37; an air passage is formed in the diversion bracket 3; the structure of the diversion cover 37 can be designed according to the needs; in this embodiment, the overall shape of the diversion cover 37 can be rhombus;
the air passage is communicated with the suction assembly; at least one suction hole 34 is provided at the position where the air passage is located at the diversion cover 37; the suction hole 34 is located at the bottom of the diversion cover 37, and the suction hole 34 is isolated from the inside of the collection assembly by the diversion cover 37. Since the diversion cover 37 protrudes from the diversion bracket 3, the isolation can be effectively completed through the diversion cover 37, and the structure of the diversion surface 33 of the diversion cover 37 allows the nasal mucus to flow smoothly.

A collection cavity 16 is formed after the collection cup mask 2 is docked with the collection cup base 4 through the diversion bracket 3, and the nasal mucus entering through the liquid inlet gap cavity 38 enters into the collection cavity 16 for collection. The collection cup mask 2 can be opened regularly to clean the collection cavity 16, which is very convenient. The diversion bracket 3 can also be disassembled and cleaned separately, and can be installed after cleaning.

As shown in FIGS. 1 and 2, the housing 100 comprises a front cover 13 and a rear cover 7, which are assembled into one body; the housing 100 is provided with a display screen lens 14; the control assembly is installed in the internal space formed by the front cover 13 and the rear cover 7, and the control assembly comprises a main board 15 and a lithium battery 8; the lithium battery 8 is used to supply power to the main board 15 and the suction assembly, and a display screen and a USB charging stand 10 are installed on the main board 15. The USB charging stand 10 is used to charge the lithium battery 8, and of course other charging stands can also be used.

In order to realize the control of components such as the air pump 9, in the embodiment, the main board 15 is also installed with a button assembly 12; the button assembly 12 can comprise function buttons and a power button, through which the power of the air pump 9 can be controlled to obtain different suction strengths; for better interactivity, in the embodiment, the housing 100 is also installed with a horn 11, and the horn 11 is connected to the main board 15 and powered by the lithium battery 8. The horn 11 can broadcast the current mode, the progress of the suction, the power, etc.; for example, after a liquid level sensor is arranged in the collection cavity 16, if the nasal mucus storage is too much, the user can be reminded to clean up through voice prompts, etc., and some music can also be played through the horn 11 to increase the entertainment effect.

In the embodiment, the suction assembly comprises an air pump 9 and an air pump connecting seat 6; the air pump 9 is connected to the main board 15, and is controlled to open and close by the main board 15; the air pump 9 is powered by the lithium battery 8; the air pump 9 is docked with the air pump connecting seat 6, and the other end of the air pump connecting seat 6 is docked and sealed with the collection cup base 4. A body decoration ring 5 is also installed outside the docking position of the air pump connecting seat 6 and the collection cup base 4, and the decorative aesthetics are increased by the body decoration ring 5.

The above are only specific embodiments of the invention, but the protection scope of the invention is not limited thereto. Any modifications or substitutions creative efforts shall all fall within protection scope of the invention. Therefore, the protection scope of the invention should be subject to the protection scope defined by the claims.

1. An anti-backflow nasal aspirator, comprising:
a housing (100), wherein a control assembly and a suction assembly are installed in the housing (100), and the suction assembly is opened or closed by the control assembly;
a collection assembly, wherein the collection assembly is hermetically installed on the housing (100), and the collection assembly is hermetically docked with the suction assembly; the collection assembly is provided with a collection cavity (16);
a suction nozzle (1), wherein the suction nozzle (1) is installed at the input end of the collection assembly; when the suction assembly generates negative pressure, it acts on the suction nozzle (1);
a diversion bracket (3), wherein the diversion bracket (3) is installed in the collection assembly; a diversion surface (33) is formed on the diversion bracket (3), and the diversion surface (33) is used for diverting nasal mucus to prevent nasal mucus from entering the airway of the suction assembly.

2. The anti-backflow nasal aspirator of claim 1, wherein the collection assembly comprises a collection cup mask (2) and a collection cup base (4); the collection cup mask (2) is connected and assembled with the collection cup base (4) in a detachable manner, and an O-ring is installed on the assembly surface; the diversion bracket (3) is installed between the collection cup mask (2) and the collection cup base (4); one end of the collection cup base (4) is connected to the suction assembly, and the other end thereof is sealed to a first docking port (32) of the diversion bracket (3); a second docking port (36) at the other end of the diversion bracket (3) is docked with the inner docking joint of the collection cup mask (2).

3. The anti-backflow nasal aspirator of claim 1, wherein the diversion bracket (3) comprises a columnar bracket body (31); one side of the bracket body (31) forms a first docking port (32), and the other side of the bracket body (31) is provided with a docking boss (35); the side of the docking boss (35) opposite to the inner docking joint forms a second docking port (36), and the second docking port (36) forms a liquid inlet gap cavity (38) after being docked with the inner docking joint.

4. The anti-backflow nasal aspirator of claim 2, wherein the outer end of the collection cup mask (2) is provided with an outer docking joint, and the outer docking joint is used for hermetically docking with the suction nozzle (1).

5. The anti-backflow nasal aspirator of claim 1, wherein the upper and lower ends of the diversion bracket (3) are also provided with a diversion cover (37), and the whole diversion cover (37) is diamond-shaped; inclined diversion surfaces (33) are formed on both sides of the diversion cover (37); an air passage is formed in the diversion bracket (3), and the air passage is communicated with the suction assembly; at least one suction hole (34) is provided at the position where the air passage is located at the diversion cover (37); the suction hole (34) is located at the bottom of the diversion cover (37), and the suction hole (34) is isolated from the inside of the collection assembly by the diversion cover (37).

6. The anti-backflow nasal aspirator of claim 2, wherein a collection cavity (16) is formed after the collection cup mask (2) is docked with the collection cup base (4) through the diversion bracket (3), and the nasal mucus entering through the liquid inlet gap cavity (38) enters into the collection cavity (16) for collection.

7. The anti-backflow nasal aspirator of claim 1, wherein the housing (100) comprises a front cover (13) and a rear cover (7), which are assembled into one body; the housing (100) is provided with a display screen lens (14); the control assembly is installed in the internal space formed by the front cover (13) and the rear cover (7), and the control assembly comprises a main board (15) and a lithium battery (8); the lithium battery (8) is used to supply power to the main board (15) and the suction assembly, and a display screen and a USB charging stand (10) are installed on the main board (15).

8. The anti-backflow nasal aspirator of claim 7, wherein the main board (15) is also installed with a button assembly (12); the housing (100) is also installed with a horn (11), and the horn (11) is connected to the main board (15) and powered by the lithium battery (8).

9. The anti-backflow nasal aspirator of claim 1, wherein the suction assembly comprises an air pump (9) and an air pump connecting seat (6); the air pump (9) is connected to the main board (15), and is controlled to open and close by the main board (15); the air pump (9) is powered by the lithium battery (8); the air pump (9) is docked with the air pump connecting seat (6), and the other end of the air pump connecting seat (6) is docked and sealed with the collection cup base (4).

10. The anti-backflow nasal aspirator of claim 9, wherein a body decoration ring (5) is also installed outside the docking position of the air pump connecting seat (6) and the collection cup base (4).
